(19) United States
(12) Patent Application Publication
(10) Pub. No.: US 2008/0131381 A1
(43) Pub. Date: Jun. 5, 2008

(54) METHODS FOR PHOTOSTABILIZING INGREDIENTS WITHIN COSMETICS, PERSONAL CARE AND HOUSEHOLD PRODUCTS AND COMPOSITIONS OBTAINED THEREFROM

(76) Inventors: Ratan K. Chaudhuri, Lincoln Park, NJ (US); Zoia Lascu, Valhalla, NY (US)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/863,472

(22) Filed: Sep. 28, 2007

Related U.S. Application Data
(60) Provisional application No. 60/847,911, filed on Sep. 29, 2006.

(51) Int. Cl. C09K 3/00; A61K 8/37; A61Q 17/04

(52) U.S. Cl. 424/59; 252/380; 512/2

(57) ABSTRACT

The present invention relates to methods and compositions for using photostabilizer compounds for stabilizing formulation ingredients, like flavors, fragrances, colors, antioxidants, polymers, within cosmetics, personal care and household products, against degradation from sun light, heat and air oxidation resulting in improvement in storage stability, viscosity, and maintenance of color of the formulated products.

This application claims the benefit of the filing date of U.S. Provisional Application Ser. No. 60/847,911, filed Sep. 29, 2007, which is incorporated by reference herein.

### FIELD OF THE INVENTION

The present invention relates to methods for using certain photostabilizer compounds in combination with UV-A and UV-B organic sunscreens. to enhance the stabilization of photosensitive formulation ingredients, like, for example, flavors, fragrances, colorants (pigments, dyes), antioxidants, vitamins, oils, waxes, UV filters, light sensitive inorganics and polymers against degradation from light, resulting in compositions with improved storage stability, viscosity, and maintenance of color.

### BACKGROUND OF THE INVENTION

Achieving photostable personal care products, household products and cosmetics is a challenge to the formulator because of inherent instabilities of certain organic compounds, like antioxidants (natural Tocopherols, Carotenoids), fragrances (Vanillin), colored organic compounds (Guaiazulene), water-soluble dyes etc. Currently available photostabilizers such as Oxynex® ST stabilizer satisfy the need to stabilize photosensitive compounds in these products. However, improvements in stabilization performance are always desired.

U.S. Published Application Number 2005/0244349, describes the use of dialkylbenzalmalonate UV filters as photostabilizers for personal care products, household products and cosmetics. The dialkylbenzalmalonate, di-(2-ethylhexyl)-3,5-dimethoxy-4 hydroxy benzylidene malonate (diethylhexyl syringylidene malonate), is described as a suitable photostabilzer and is commercially available under the tradename Oxynex® ST. These dialkylbenzalmalonate compounds are also known to have sunscreen activity, i.e., they have a chromophoric moiety which absorbs within the ultra violet range from 290-400 nm, see U.S. Pat. Nos. 6,602,515 and 6,831,191.

Organic sunscreens are classified as UV-A filters, UV-B filters or broad spectrum filters (UV-A and UV-B functionality in a single molecule) depending on the type of radiation they absorb. UV-A sunscreens absorb radiation in the 320 to 400 nm regions of the ultra violet spectrum and UV-B sunscreens absorb radiation in the 290 to 320 nm regions of the ultra violet spectrum. Broad band sunscreens (UV-A and UV-B functionality) absorb radiation in the 290 to 400 nm region of the ultra violet spectrum and have two maximums, one in the UV-B region and the other in the UV-A region.

Representative references related to UV sunscreens are:

U.S. Pat. No. 3,278,448, which discloses cinnamic acid derivatives such as 4-hydroxy, 3-5-ditertbutyl-alphacarbethoxy-cinnamic acid ether ester in column 2, line 20;

U.S. Pat. No. 3,538,226, which describes cinnamic acid alkyl ester derivatives at column 1, lines 15-31 and column 2, lines 1-12 and column 3, lines 30-55 and 60;

U.S. Pat. No. 5,175,340, which describes cinnamic acid alkyl esters having hydroxy radicals and alkoxy radicals on the phenyl ring, and

U.S. Pat. No. 6,436,377, which describes UV-A filters that are salicylate derivatives; and

U.S. Pat. No. 7,094,432, describes UV-B filters that are dibenzoyl methane derivatives such as Avobenzone.

### SUMMARY OF THE INVENTION

Many ingredients in cosmetics, personal care products and household products are not always photochemically stable, which limits the utility of these compositions. Examples of photosensitive ingredients are poly-unsaturated and aromatic compounds, which include colorants, antioxidants, flavors, vitamins, U.V. filters and fragrances. More specific examples include carotenoids, tocopherols, guaiazulene, vanillin and menthylanthranillate. Other photosensitive ingredients which need not have unsaturated or aromatic structure include polymeric thickening agents, oils, waxes, certain organic dyes and certain inorganic compounds used in cosmetics or personal or household products.

It has been found that dialkylbenzalmalonate photostabilizers, such as Oxynex® ST photostabilzer, when combined with a photo-unstable UV-A organic sunscreen such as Avobenzone and a UV-B organic sunscreen such as Homosalate, Octylsalate, Octylemethoxy cinnamate, Octocrylene provide a superior stabilization of photosensitive ingredients under UV-light.

It has now been discovered that the stabilization of photo-sensitive ingredients within formulations such as cosmetics, personal care products and household products provided by the dialkylbenzalmalonates can be enhanced by combining these dialkylbenzalmalonates with a UV-A filter and a UV-B filter. This combination of agents comprises at least one of each of the following agents, a dialkylbenzalmalonate, a UV-A screen and a UV-B sunscreen.

Preferred dialkylbenzalmalonates are of formulae I and II:

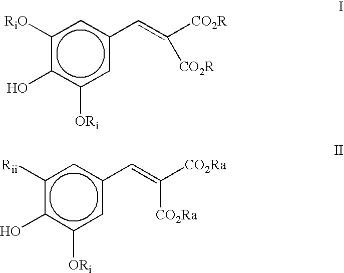

For formulae I, each Rᵢ is independently linear or branched C₁-C₈ alkyl and each R is independently linear or branched C₁ to C₂₀ alkyl

For formulae II, Rᵢ is a linear or branched C₁-C₈ alkyl Rᵢᵢ is hydrogen or linear or branched C₁-C₈ alkyl and each Ra is independently, linear or branched C₁ to C₃₀ alkyl.

Included within the preferred compounds are those of formula I wherein each Rᵢ is independently linear or branched C₁-C₄ alkyl and each R is independently linear or branched C₁-C₁₂ alkyl.

Included within the preferred compounds are those of formula II wherein Rᵢ and Rᵢᵢ are independently linear or branched C₁-C₄ alkyl, and each Rₐ is independently linear or branched C₁-C₁₂ alkyl.

While compounds having from C₁-C₈ alkyl groups for R and Rₐ of formula I and II are preferred, significant utility can be obtained from compounds wherein R and Rₐ are linear or branched C₉ to C₂₀ alkyl groups.

Another preferred class of compounds is those of formulae III, IV, V and VI

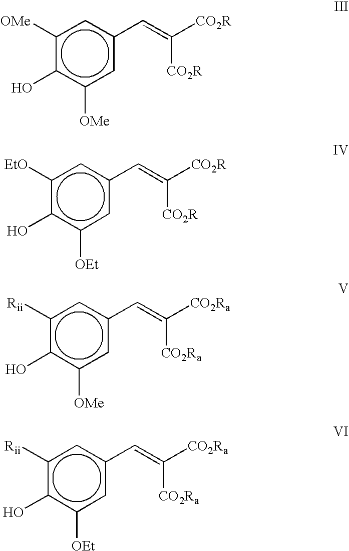

wherein

R is linear or branched C₁ to C₂₀ alkyl,

Rₐ is linear or branched C₁ to C₃₀ alkyl, and

Rᵢᵢ is linear or branched C₁-C₈ alkyl or hydrogen.

Preferred compounds of Formulae I, II, III and IV, include:
diethyl-3,5-dimethoxy-4-hydroxy benzylidene malonate,
di-(2-ethylhexyl)-3,5-dimethoxy-4-hydroxy benzylidene malonate,
diisoamyl-3,5-dimethoxy-4-hydroxy benzylidene malonate,
didodecyl-3,5-dimethoxy-4-hydroxy benzylidene malonate,
dipalmitoyl-3,5-dimethoxy-4-hydroxy benzylidene malonate, and
di-isopropyl-3,5-dimethoxy-4-hydroxy benzylidene malonate.

Preferred compounds of Formulae V and VI include:
diethyl-3-methoxy-4-hydroxy benzylidene malonate,
di-(2-ethylhexyl)-3-methoxy-4-hydroxy benzylidene malonate,
diisoamyl-3-methoxy-4-hydroxy benzylidene malonate,
didodecyl-3-methoxy-4-hydroxy benzylidene malonate,
dipalmitoyl-3-methoxy-4-hydroxy benzylidene malonate, and
di-isopropyl-3-methoxy-4-hydroxy benzylidene malonate.

Any conventional UV-A organic sun screen is suitable for use the combinations employed in this invention but preferred compounds belonging to the class of dibenzoylmethane derivatives. Examples of such compounds are as follows: 2-methyldibenzoylmethane, 4-methyldibenzoylmethane, 4-isopropyldibenzoylmethane, 4-tert-butyldibenzoylmethane, 2,4-dimethyldibenzoylmethane, 2,5-dimethyldibenzoylmethane, 4,4′-diisopropyldibenzoylmethane, 4,4′-dimethoxydibenzoylmethane, 4-tert-butyl-4′-methoxydibenzoylmethane, 2-methyl-5-isopropyl-4′-methoxydibenzoylmethane, 2-methyl-5-tert-butyl-4′-methoxydibenzoylmethane, 2,4-dimethyl-4′-methoxydibenzoylmethane, and/or 2,6-dimethyl-4-tert-butyl-4′-methoxydibenzoylmethane). A preferred UV-A organic sunscreen is Avobenzone.

Further examples of UV-A sunscreens can be found in the chapter article entitled, “Regulations of Sunscreens Worldwide” written by D C Steinberg, in Sunscreens: Regulations and Commercial Development, Third Edition, N A Shaath, Ed, Tayler & Francis, 2005.

Any conventional UV-B sun screen is suitable for use in the combinations employed in this invention. Suitable UV-B filters include aminobenzoic acid, cinoxate, dioxybenzone, oxybenzone, padimate (0), salicylate derivatives such as homosalate, octocrylene, octyl methoxy cinnamate, phenylbenzimidazole sulfonic acid, sulisobenzone and trolamine salicylate. Preferred UV-B sunscreens are the salicylate, cinammate, or cyanoacrylate derivatives. These derivatives are generally of formula X, Y and Z, respectively, below.

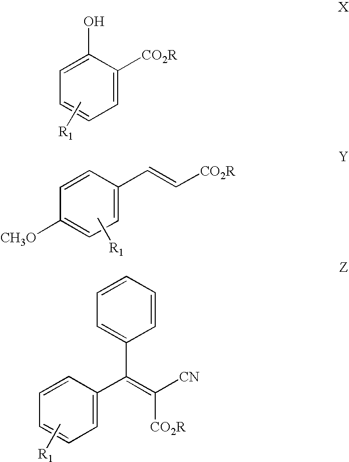

wherein R is linear or branched C₁ to C₂₀ alkyl and R′ is hydrogen or a linear or branched C₁ to C₈ alkyl. Preferred compounds are octyl salicylate, homosalate, triethane amine (TEA)-salicylate, diethylamine (DEA)-salicylate, Octylymethoxy cinammate, Isoamylmethoxy cinnamate and Octocrylene.

Further examples of UV-B sunscreens can be found in the chapter article entitled, “Regulations of Sunscreens Worldwide” written by D C Steinberg, in Sunscreens: Regulations and Commercial Development, Third Edition, N A Shaath, Ed, Tayler & Francis, 2005.

Optionally, this inventive composition may contain, emollients, such as, caprylic/capric triglycerides, cocotriglycerides, dibutyladipate, dicapryl carbonate, C-12-15 alkyl benzoate, cyclomethicone, dicaprylether, etc.

A method provided by this invention comprises combining at least one of each of the three agents discussed above with at least one photosensitive compound which is either a colorant (dye, pigment), a fragrance, a flavoring agent, vitamin, UV filter, antioxidant or a combination of these compounds (color/fragrance/flavor/antioxidant). Additionally, the use of photosensitive polymeric compounds, oils, waxes, and certain inorganics, which may not have any aromatic or poly-unsaturated functionality, is also part of this invention.

In another aspect, a method is provided for stabilizing at least one photosensitive compound which is either a colorant (dye, pigment), a fragrance, a flavoring agent, vitamin, UV filter, antioxidant, oil, wax, polymer, photosensitive inorganic pigment or a combination of these compounds. This method comprises combining at least one of each of the three agents discussed above with said at least one photosensitive compound in a ratio within a range of 20:1 to 1:5, preferably 10:1 to 1:1 or 1:2, more preferably 5:1 to 2:1 or 1:1, based on total weight percent of the three agents discussed above to the total weight percent of the one or more photosensitive poly-unsaturated and/or aromatic compounds.

Stabilizing compounds that are photosensitive in the context of this invention means reducing the degradation of these photosensitive compounds, which have absorbed visible light or UV rays to form high energy chromophores. Thus, the phrase “photosensitive in the visible light range” refers to compounds that form unstable chromophores once visible light is absorbed which may or may not be stable once UV radiation is absorbed. Accordingly, compounds or compositions that are only exposed to visible light, or substantially or predominantly only to visible light, for example, during storage in a warehouse, on a shelf in a store or in a home, are protected. Protection continues even when protected compounds by the methods of the invention are exposed to natural sunlight that contains both UV and visible light.

In preferred embodiments, combinations according to the invention are formed within or transferred to a cosmetic composition, personal care product or household product. In a further preferred embodiment, the cosmetic composition, personal care product or household product is within a container for retail sale when the combination is formed or transferred to a container for retail sale after the combination is formed.

The total weight percent of the three agents discussed above in the cosmetic composition, personal care product or household product is 0.001-10.0 wt %, preferably 0.01-4.0 wt %, more preferably 0.01-2.0 wt % even more preferably 0.1-1.0 wt %, and most preferably 0.02-0.5 wt %.

In other preferred embodiments, however, one or more of the photosensitive compounds is/are brought together with at least one of each of the three agents discussed above without substantially any other ingredients (other ingredients however may be present). The stabilizer combinations of the invention are effective in compositions that additionally contain only a photosensitive compound. The weight ratio of the total weight of the three agents to a photosensitive compound, in these compositions is as described above.

In other aspects, vitamins can likewise be stabilized by a combination of the three discussed above. All vitamins are included and their derivatives as they are subject to photodegradation. Vitamins and vitamin derivatives are, for example, vitamin A, vitamin A propionate, vitamin A palmitate, vitamin A acetate, retinol, vitamin B, thiamine chloride hydrochloride (vitamin B₁), riboflavin (vitamin B₂), nicotinamide, vitamin C and derivatives (for example ascorbyl palmitate, magnesium ascorbyl phosphate, ascorbyl acetate), vitamin D, ergocalciferol (vitamin D₂), vitamin E, DL-α-tocopherol, tocopherol E acetate, tocopherol hydrogensuccinate, vitamin K₁, esculin (vitamin P active ingredient), thiamine (vitamin B₁), nicotinic acid (niacin), pyridoxine, pyridoxal, pyridoxamine, (vitamin B₆), pantothenic acid, biotin, folic acid and cobalamine (vitamin B₁₂). Preferred vitamins are, for example, vitamin A palmitate, vitamin C and derivatives thereof, DL-α-tocopherol, tocopherol E acetate, nicotinic acid, pantothenic acid and biotin. Vitamin E, which is often added to cosmetic products and personal care products is also preferably stabilized by the compounds according to the invention. Additional preferred vitamins are Vitamin C and K and derivatives thereof.

Furthermore, oils and waxes can also be stabilized by a combination of the three agents discussed above. Both natural and synthetic oils and waxes can be stabilized. Natural waxes include, for example, carnauba wax, candelilla wax, rice bran wax, bee's wax, lanolin, motan wax and ceresine wax. Synthetic waxes derived from hydrocarbons are, for example, paraffin wax, microcrystalline waxes, lauric acid, myristic acid, palmitic acid, stearic acid and behenic acid, sorbitane fatty acid esters and amides. Mixtures of waxes can also be stabilized. Natural oils include, for example, coconut oil, canola oil, soybean oil, rapeseed oil, palm kernel oil, murumuru tallow, and tucum oil.

As used herein, natural waxes and oils mean any wax or oil derived from plant or animal material including, but not limited to, waxes and oils that are derived from plants that have been genetically modified either through traditional breeding or through genetic engineering techniques.

The invention also relates to the stabilization of UV protectants/filters, for example, of ethylhexyl methoxycinnamate with the combination of three agents discussed above.

The invention also includes cosmetics, personal care products and household products themselves that contain at least one photosensitive compound, which is either a colorant (dye, pigment), a fragrance, a flavoring agent, vitamin, oil, wax, antioxidant, polymeric thickener, U.V. filter, photosensitive inorganic compound or a combination of these compounds in combination at least one of each of the three agents discussed above. The household products, cosmetics and personal care products described above are protected from both visible and UV light, including from only visible light, or substantially or predominantly only from visible light, e.g., during storage, for example.

Household products are, for example, cleaning compositions, detergents, dishwashing liquids or powders, glass or furniture cleaning and/or polishing compositions, floor cleaning and/or polishing compositions, etc. The household formulations can be in the form of solids sticks, solutions, sprays, foams, liquid dispersions, or loose powders. The personal care and cosmetics formulations can be in the form of creams, ointments, suspensions, powders, oil, lotions, oleo alcoholic lotions, fatty gels, oleo-alcoholic gels and lotions, solid sticks, foams, emulsions, liquid dispersions, sprays and aerosols. More specific forms include: lotions, lipsticks, foundations, makeup, loose or press powder, eye blush, eye shadow, shampoo, conditioner and nail lacquer.

Poly-unsaturated color compounds which are not photochemically stable include, for example, carotenoids, Ubiquinones and Azulenes such as guaizulene and water based dyes. Guaiazulene has very limited photostability. Almost 90% of Guaiazulene is lost when exposed to solar simulator at a total energy of 100 mJ/cm². β-carotene, degrades in ambient light at room temperature storage.

Examples of photo-sensitive carotenoids include, for example, Lycopene, Zeaxanthine, Cantaxanthine, α-, β-, γ- & δ-Carotenes, Astacin, Astaxanthin, Chrysanthemaxanthin, Torularhodin, Violaxanthin, Capsanthin, Capsorubin and others. 1. Examples of Carotenoids—etc.

Examples of photo-sensitive Azulenes, include, for example, Azulene, Guaiazulene, Guaiol and others.

Examples of photo-sensitive Ubiquinones (Coenzyme Q), include, for example, structures based on the 2,3-dimethoxy-5-methyl-benzoquinone nucleus with a variable terpenoid side chain containing one to twelve mono-unsaturated trans-isoprenoid units with ten units being the most common in animals. Compounds can be described as Coenzyme Qₙ in which n=1-12. Naturally occurring members are the Coenzymes Q₆-Q₁₀.

Examples of tocopherols are natural tocopherols. Tocopherols are a mixture of four lipid—soluble tocopherols (α, β, γ and δ) and four lipid-soluble tocotrienols (α, β, γ and δ). Tocopherols and tocotrienols differ only in their phenyl side chain. The chromanol head of each is identical with α, β, γ and δ-isomers, each containing an essential hydroxyl group necessary for antioxidant activity. Synthetic DL-Tocopherols or its derivatives, like acetate, succinate, etc. are also included. Photochemically, tocopherols (synthetic or natural) are not very stable (R K Chaudhuri, Phyllanthus tannins, in P Elsner and H I Maibach, eds. Cosmeceuticals: Drugs vs Cosmetics, New York, Marcel and Dekker, in press).

Included within the methods of this invention is the use of the combination of three described above in stabilizing photosensitive dyes and/or organic pigments. An example of photosensitive organic pigments that are stabilized by the combination of three described above are the curcuminoids. Curcuminoids are polyphenolic pigments found in the spice turmeric. Curcuminoids are responsible for the yellow color of turmeric, as well as the yellow color of curry. The major curcuminoids are curcumin, demethoxycurcumin and bisdemethoxycurcumin. These substances comprise 3 to 6% of Curcuma longa. Curcumin makes up 70 to 75% of the curcuminoids, demethoxycurcumin 15 to 20% and bisdemethoxycurcumin about 3%. Curcuma longa is a tropical plant native to south and southeast tropical Asia. It is a member of the ginger of Zingiberaceae family. Other Curcuminoids include Cassumunin A and Cassumunin B, isolated from tropical ginger, Zingiber cassumunar.

The term turmeric is used both for the plant Curcuma longa L. and the spice derived from the rhizomes of the plant. Turmeric is widely consumed in the countries of origin for a variety of uses, including use as a dietary spice, as a dietary pigment and as an Indian folk medicine for the treatment of various illnesses.

Fragrance ingredients are, for example: Abbarome 011®, Acalea, Allyl Amyl Glycolate, alpha-Terpineol, Alpha Pinene, Ambrettolide, Amyl Cinnamic Aldehyde, Amyl Phenyl Acetate, Amyl Salicylate, Andrane, Anethole 21/22, Anethole USP, Anethole USP, Aphermate, Apo Patchone, Bacdanol®, Benzyl n-Butyrate, Benzyl Propionate, Benzyl Salicylate, Bergamal, Beta Naphthyl Isobutyl Ether, Beta Pinene Coeur, Bicyclononalactone, Bornafix®, Canthoxal, Cashmeran®, Cedrafix, Cedramber®, Cedrenyl Acetate Chinese, Celestolide, Cinnamalva, cis-3-Hexenyl Salicylate, Citral Dimethyl Acetal, Citralva®, Citronalva, Citronellol 700 98TA, Citronellol 750, Citronellol 950, Citronellol Coeur, Citronellyl Acetate, Citronellyl Acetate A, Citronellyl Acetate Pure, Citronellyl Formate, Clarycet, Clonal, Coniferan, Cyclabute, Cyclacet™, Cyclaprop™, Cyclemone A, Cyclogalbaniff, Cyclohexyl Ethyl Acetate, Cyclohexyl Ethyl Alcohol, Damascol 4, Decyl Methyl Ether, Delta Damascone, Dihydro Cyclacet, Dihydro Floralate, Dihydro Floralol, Dihydro Myrcenyl Acetate, Dihydro Terpineol, Dihydro Terpinyl Acetate, Dihydro Terpinyl Acetate DSA, Dimethyl Benzyl Carbinol, Dimethyl Benzyl Carbinyl Acetate, Dimethyl Benzyl Carbinyl Butyrate, Dimethyl Cyclormol, Dimethyl Octanol-PQ, Dimethyl Phenyl Ethyl Carbinyl Acetate, Dimyrcetol, Diola, Dipentene 5100, Dulcinyl® Recrystallized, Ethyl 3 Phenyl Glycidate, Ethyl Ortho Methoxy Benzoate, Fleuramone, Fleuranil, Floralate, Floralol, Floralozone, Fraistone, Fructone, Galaxolide® 50 BB, Galaxolide® 50 DEP, Galaxolide® 50 DPG, Galaxolide® 50 IPM, Galbanum Coeur, Gelsone, Geraldehyde, Geraniol 5020, Geraniol 7030®, Geraniol 980, Geraniol Coeur, Geranyl Acetate, Geranyl Acetate Extra, Geranyl Acetate Pure, Grisalva, Guaiyl Acetate, Helional™, Herbac, Hexadecanolide, Hexylon, Hexyl Acetate, Hexyl Cinnamic Aldehyde, Hexyl Salicylate, Hyacinth Body, Hyacinth Body No. 3, Hydratropic Aldehyde Dimethyl Acetal, Hydroxyol, Hypo-Lem, Indolarome, Indolene 50, Intreleven Aldehyde, Intreleven Aldehyde Special, Ionone 100%, lonone Alpha, lonone Alpha Beta, Regular, Ionone Beta, Iso Amyl Butyrate, Iso Bornyl Propionate, Iso Butyl Quinoline, Iso E Super®, Isoamyl Salicylate, Isobutyl Phenyl Acetate, Isocyclemone E, Isocyclocitral, Isocyclogeraniol, Isoproxen, Jasmal, Jasmelia, Jessemal™, Kharismal, Koavone®, Kohinool®, Lavonax, Lemsyn, Liffarome, Lindenol®, Lymolene, Lyral®, Lyrame, Lyrame Super, Maritima, Melafleur, Methyl Anthranilate, Methyl Cedryl Ketone Chinese, Methyl Cinnamic Aldehyde alpha, Methyl Ionone Gamma A, Methyl lonone Gamma Coeur, Methyl lonone Gamma Pure, Methyl Lavender Ketone, Muguesia, Muguet Aldehyde 50, Muguet Aldehyde 50 BB, Myrac Aldehyde, Myrcenol Super, Myrcenyl Acetate, Neoproxen, Nerol 800, Nerol 850, Nerol 900, Neryl Acetate, Ocimene, Ocimenyl Acetate, Octacetal, Orange Flower Ether, Orivone, Orriniff 25% IPM, Oxaspirane, Ozofleur, Pamplefleur®, Peomosa, Phenafleur, Phenoxanol®, Phenoxyethyl Isobutyrate, Phenoxyethyl Propionate, Phenyl Ethyl Acetate, Phenyl Ethyl Alcohol, Phenyl Ethyl Benzoate, Phenyl Ethyl Formate, Phenyl Ethyl Isobutyrate, Phenyl Ethyl Phenyl Acetate, Phenyl Ethyl Salicylate, Piconia, Precyclemone B, Prenyl Acetate, Proflora, Pseudo Linalyl Acetate, Reseda Body, Rosalva, Rosamusk, Roseate, Rosemarel, Salicynalva, Sanjinol, Santaliff, Spirodecane, Strawberiff®, Styrallyl Propionate, Syvertal, Terpineol 900, Terpineol Extra, Terpinolene 20, Terpinolene 90, Terpinolene 90 PQ, Terpinyl Acetate (CST), Terpinyl Acetate (GUM), Tetrahydro Geraniol, Tetrahydro Muguol, Tetrahydro Muguol Coeur, Tetrahydro Myrcenol, Tetrameran, Tobacarol, Trimofix® 0, Triplal™, Triplal™ Extra, Unipine 60®, Unipine 75®, Unipine 80®, Unipine 85, Unipine 90®, Unipine NCL®, Unipine S-70®, Unitene D®, Unitene LP®, Unitene WST®, Vandor B, Vanoris, Verdol, Verdox™, Verdox™ HC, Verdural B Extra, Verdural Extra, Vernol®, Vertenex®, Vertenex® HC, Vertofix® Coeur, Vigoflor, and Violiff.

Preferred fragrance ingredients are, for example, Muguet Aldehyde 50, Myrac Aldehyde, Phenyl Ethyl Formate, Precyclemone B, Syvertal, Strawberiff®, and Triplal™. The chemical formulae for these compounds, in the order listed across are:

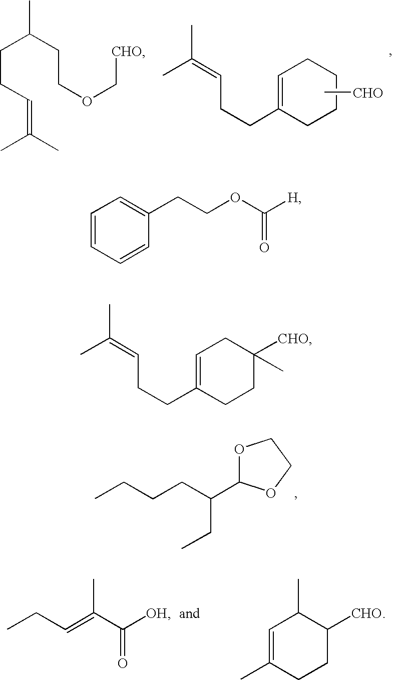

Flavor ingredients are, for example: 4,5-Dimethyl-2-ethyl-3-thiazoline, 6-Methyl Coumarin, Allyl Caproate, Anethole USP, Asafoetida Oil English Distilled SAS, Black Pepper, Black Pepper, Black Pepper Oil, Black Pepper Oil English Distilled SAS, Buchu Sulfur Fractions, Butyric Acid, Cardamon Oil English Distilled SAS, Cassia Oil, Cassia Oil Redistilled, Cinnamon Bark Oil, Cinnamon Leaf Oil Cleaned, Clove Bud Oil English Distilled SAS, Clove Leaf Oil Cleaned, Clove Leaf Oil Redistilled, Cocal™, Cocoa Distillate (Nat.), Cocoa Essence Dark, Cocoa Essence White, Cocoa Extract Nat., Coffee Enhancer Base, Coffee Enhancer W/S, Coffee Extract, Coffee Extract Italian Roast M3881 Nat., Coffee Extract Nce Iiim Nat., Coffee Extract Nce Iv Nat., Coriander Oil, Cyclodithalfarol-705, delta Decalactone, Dimethyl Benzyl Carbinyl Butyrate, Dimethyl Sulfide, Dithione 865, Ethyl-2-Methyl Butyrate, Ethyl-3-Hydroxy Butyrate, Ethyl Butyrate, Ethyl Iso Butyrate, Ethyl Iso Valerate, Ethyl Oxanoate 369, Eucalyptus Oil 80%, Farnesene 1% PG/ETOH, Furfurrole 302, gamma-Decalactone, gamma-Hexylactone, gamma-Octalactone, gamma Dodecalactone, Ginger Oil Chinese, Ginger Oil Nigerian English Distilled SAS, Grapefruit Key, Grill Flavor O/S, Grill Flavor W/D, Heptan-2-One (Nat.), Hexene-3-One-4, Hexyl Acetate, Homo Cyclocitral, beta, Honey Distillate Nat., Ionone Beta, Iso Amyl Iso Valerate, Iso Butyl Caproate, Iso Fragarone-030, Iso Fragarone, 1% ETOH™, Isobutyl Furyl Propionate, Isovaleric Acid, Juniperberry Oil English Distilled SAS, Ketone Mix, Kumarone™, Lemon Oil 5X Sas, Lemon Oil Terpeneless Sas, Lemonless Lemon Key, Lime Oil Terpeneless, Linalool 75/80% Ex Orange (Nat.), Linalyl Acetate (Nat.), Mangone 5% ETOH™, Methional, Methyl Butyric Acid (2), Methyl Ketones (Nat.), Methyl Oxycyclosulfide 719, Mushroom Extract, Natural Flavor (99% Vanillin), Nat. Cocoa Butter Distillate, Nat. Peanut Distillate, Nonan-2-One (Nat.), Nutmeg Oil East Indian, Octanal 35% (Nat.), Octen-4-one-2, Olibanum Oil English Distilled SAS, Orange Oil 15X Decolorized M3706, Orange Oil 950 (10X), Orange Oil Terpeneless 2501, Oxaromate-884, Oxycyclothione-030, Paradiff™ 0.01% ETOHGR, Paradiff™ 0.01% Grapefruit Oil, Peach Flavor Key, Peppermint Oil Redistilled Yakima, Peppermint Oil Spec. Fractions, Phenyl Ethyl 2-Methyl Butyrate, Phenyl Ethyl Acetate, Phenyl Ethyl Alcohol, Phenyl Ethyl Isovalerate, Phenyl Oxaromate-681, Pimento Berry Oil English Distilled SAS, Pimento Leaf Oil, Pimento Leaf Oil Cleaned, Pineapple Compound 15% ETOH GR, Pineapple Compound 15% PG, Popcorn Chemical, Propionic Acid, Raspberry Flavor Key, Raspberry Flavor Key, Raspberry Flavor Key, Robustone 1.0% ETOH™, Robustone™, Schinus Molle Oil, Sclareolide, Sesame Distillate Nat., Sinensals (Nat.), Spearmint Oil Terpeneless, Starter Distillate 15X W/S, Strawberriff, Strawberry Base, Strawberry Flavor Key, Strawberry Flavor Key, Succinic Acid, Sulfurome-015, Sweetness Modifier, Tetrahydro Terrazine-014™, Thionol-935, Thionol-966, trans-2-Hexenal, Trimenal Acetate 399 1% ETOH™, Tropical Fruit Key Base, Tropical Fruit Key Base, Undecan-2-One (Nat.), Varamol-106 10% ETOH, Varamol-106 10% NEBM5, and Varamol-106 10% PG.

Preferred flavor ingredients are, for example, Methional, whose chemical formula is

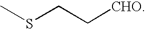

Essential oils can also be photostabilized by the combination of agents employed in this invention. The INCI Dictionary includes more than 100 essential oils. Only a few of them are mentioned here: Basil, Bergamot, Black Pepper, Cedarwood, Chamomile, Cinnamon, Clary Sage, Clove, Coriander, Cypress, Eucalyptus, Fennel, Geranium, Ginger, Grapefruit, Jasmine, Juniper, Lavender, Lemon, Lemongrass, Melaleuca or Tea Tree, Myrrh, Olibanum, Patchouli, Peppermint, Rose, Rosemary, Rosewood, Sage, Sandalwood, and Thyme.

Essential oils can be obtained, for example, by compression or solvent extraction. In an alternate process, odoriferous plant parts are steam distilled. The oil may then be collected by separation from the condensed distillate. The leftover water distillate contains plant components and some volatile oils. This material is normally marketed as a water. Typical flower representatives are cabbage rose (Rosa centifolia) flower water, matricaria water, and peppermint leaf water. A similar distillate is obtained from Hamamelis virginiana but is marketed primarily as a hydroalcoholic solution.

Natural colors, compounds responsible for the coloration of plants, etc., appearing in nature, e.g., extracts thereof or synthetic versions thereof, can also be stabilized, i.e., protected from photodegradation, by combinations of three agents used in this invention (which compounds can be unstable) and methods which employ them.

Certain pigments, for example, Annatto (Bixin), Anthocyanin, Beta-carotene, Betanin, Capsanthin/capsorubin, Chlorophyll, Crocetin, Curcumin, and Luteolin, can also be photostabilized by the combinations and methods of this invention.

Bioflavonoids and flavonoids can also be photostabilized by the combinations and methods of this invention. These compounds belong to a large series of plant-derived phenolics. Some of these compounds are deeply colored and may be used for their tinctorial attributes. Some members of this group impart color. Compounds belonging to this group of compounds, are, for example, Alizarin, Purpurin, Amaranth, Annatto, Anthocyanidins, Apigenin, Azulene, Betalaines and Betanines, phytolaccanin, (Blue) Gardenia, Caramel, Carotenes, Lycopene, Canthaxathin, Capsanthin/Capsorubin, Xanthophyll, Carthamin, Chlorophyll, Crocin and Crocetin, crocetin, Curcumin, desmethoxycurcumin, Indigo, Juglone, Lawsone, Luteolin, Phycocyanobilin, Pratol, Santalin, Shikonin, and alkannin.

Certain inorganic compounds also photodegrade, and can be stabilized against this degradation by the combinations and methods of this invention. Some exemplary inorganic compounds are: iron oxides, which may be represented by the general formulas as follows: Yellow Oxide Fe₂O₃—H₂O. Red Oxide, being the anhydrous form of Yellow Oxide, is Fe₂O₃. Black Oxide is FeO—Fe₂O₃; Ferric ammonium ferrocyanide may be represented as Fe(NH₄)Fe(CN)₆.nH₂O; Manganese violet, a manganese ammonium pyrophosphate complex, may be represented as Mn(NH₄)P₂O₇; Ultramarine blue is a complex sodium aluminum sulfo-silicate having the approximate formula Na₇Al₆O₂₄S₃. The intense color is the result of the polysulfide linkage, which is present in a highly resonant state; Chrome oxide green, is a chromium sesquioxide Cr₂O₃, whereas hydrated chromium sesquioxide is principally Cr₂O₃.nH₂O.

Some organic colors can also be photostabilized by the combinations and methods of this invention. For example, reds (D&C Red #6 Barium Lake, D&C Red #6, D&C Red #7, D&C Red #21, D&C Red #22, D&C Red #27, D&C Red #28, D&C Red #30, D&C Red #33 Aluminum Lake, D&C Red #34 Calcium Lake, D&C Red #36, and FD&C Red #40 Aluminum Lake), orange (D&C Orange #5), yellows (FD&C Yellow #5 Aluminum Lake, FD&C Yellow #6 Aluminum Lake, FD&C Yellow #10 Aluminum Lake), and blue (FD&C Blue#1 Aluminum Lake).

A listing of approved colorants (except hair colors) that can be stabilized by the compounds of the invention are found in U.S. Food and Drug Administration color additive regulations, 21 CFR 73 and 74. A listing of colorants in EU Member States may be found in Annex IV of the EC Cosmetics Directive 76/768/EEC.

Some colorants are, for example, Acid Black 1, Acid Black 52, Acid Blue 3, Acid Blue 9, Acid Blue 9 Aluminum Lake, Aka223, Chromium Oxide Greens, Acid Blue 9 Ammonium Salt, Acid Blue 62, Acid Blue 74, Acid Blue 74 Aluminum Lake, Acid Green 1, Acid Green 25, Acid Green 50, Acid Orange 6, Acid Orange 7, Acid Red 14 Aluminum Lake, Acid Red 18, Acid Red 18 Aluminum Lake, Acid Red 27, Acid Red 27 Aluminum Lake, Acid Red 33, Acid Red 51, Acid Red 73, Acid Red 87, Acid Red 92, Acid Red 95, Acid Red 184, Acid Red 195, Acid Violet 9, Acid Violet 43, Acid Yellow 3, Acid Yellow 3 Aluminum Lake, Acid Yellow 23, Acid Yellow 23 Aluminum Lake, Acid Yellow 73, Basic Blue 6, Acid Yellow 73 Sodium Salt, Acrylic Resin Coated Aluminum Powder, Aka2, Aka3, Aka102, Aka104(1), Aka105(1), Aka106, Aka201, Aka202, Aka203, Aka204, Aka205, Aka206, Aka207, Aka20 8, Aka213, Aka214, Aka215, Aka218, Aka219, Aka220, Aka221, Aka22, Aka225, Aka226, Aka227, Aka228, Aka230(1), Aka230(2), Aka231, Aka232, Aka401, Aka404, Aka405, Aka500, Aka502, Aka503, Aka504, Aka505, Aluminum Laccate, Aluminum Powder, Aluminum Stearate, Annatto, Anthocyanins, Ao1, Ao2, Ao201, Ao202, Ao203, Ao204, Ao205, Ao403, Ao404, Astaxanthin, Basic Blue 6, Basic Blue 41, Basic Yellow 11, Beetroot, Bismuth Oxychloride, Blue 1, Blue 1 Lake, Blue 4, Brilliant Black 1, Bromocresol Green, Bromothymol Blue, Bronze Powder, Brown 1, Calcium Ferrite, Calcium Stearate, Capsanthin/Capsorubin, Caramel, Carbon Black, Carmine, Beta-Carotene, Carotenolds, Chlorophyllin-Copper Complex, Chromium, Chromium Hydroxide Green, Cl 10006, Cl 10020, Cl 10316, Cl 11680, Cl 11710, Cl 11725, Cl 11920, Cl 12010, Cl 12085, Cl 12120, Cl 12150, Cl 12370, Cl 12420, Cl 12480, Cl 12490, Cl 12700, Cl 13015, Cl 14270, Cl 14700, Cl 14720, Cl 14815, Cl 15510, Cl 15525, Cl 15580, Cl 15620, Cl 15630, Cl 15800, Cl 15850, Cl 15865, Cl 15880, Cl 15980, Cl 15985, Cl 16035, Cl 16185, Cl 16230, Cl 16255, Cl 16290, Cl 17200, Cl 18050, Cl 18130, Cl 18690, Cl 18736, Cl 18820, Cl 18965, Cl 19140, Cl 20040, Cl 20170, Cl 20470, Cl 21100, Cl 21108, Cl 21230, Cl 24790, Cl 26100, Cl 27290, Cl 27755, Cl 28440, Cl 40215, Cl 40800, Cl 40820, Cl 40825, Cl 40850, Cl 42045, Cl 42051, Cl 42053, Cl 42080, Cl 42090, Cl 42100, Cl 42170, Cl 42510, Cl 42520, Cl 42735, Cl 44045, Cl 44090, Cl 45100, Cl 45190, Cl 45220, Cl 45350, Cl 45370, Cl 45380, Cl 45396, Cl 45405, Cl 45410, Cl 45425, Cl 45430, Cl 47000, Cl 47005, Cl 50325, Cl 50420, Cl 51319, Cl 58000, Cl 59040, Cl 60724, Cl 60725, Cl 60730, Cl 61565, Cl 61570, Cl 61585, Cl 62045, Cl 69800, Cl 69825, Cl 71105, Cl 73000, Cl 73015, Cl 73360, Cl 73385, Cl 73900, Cl 73915, Cl 74100, Cl 74160, Cl 74180, Cl 74260, Cl 75100, Cl 75120, Cl 75125, Cl 75130, Cl 75135, Cl 75170, Cl 75300, Cl 75470, Cl 75810, Cl 77000, Cl 77002, Cl 77004, Cl 77007, Cl 77015, Cl 77120, Cl 77163, Cl 77220, Cl 77231, Cl 77266, Cl 77267, Cl 77268:1, Cl 77288, Cl 77289, Cl 77346, Cl 77400, Cl 77480, Cl 77489, Cl 77491, Cl 77492, Cl 77499, Cl 77510, Cl 77713, Cl 77742, Cl 77745, Cl 77820, Cl 77891, Cl 77947, Cobalt Aluminum Oxide, Cochineal, Copper Powder, Crocus Sativus Flower Extract, Curry Red, Daidai201, Daidai203, Daidai204, Daidai205, Daidai206, Daidai207, Daidai401, Daidai402, Daidai403, Dihydroxyacetone, Direct BLue 86, Disodium EDTA-Copper, Dunaliella Bardawil Powder, Epoxy Resin Coated Aluminum Powder, Erythrulose, Ext. Violet 2, Ext. Yellow 7, Ext. Yellow 7 Lake, Fast Green FCF, Ferric Ammonium Citrate, Ferric Ammonium Ferrocyanide, Ferric Ferrocyanide, Fluorescent Brightener 230, Fluorescent Brightener 236, Gardenia Florida Extract, Gold, Green 3, Green 3 Lake, Green 5, Green 6, Green 8, Guaiazulene, Guanine, Haematococcus Pluvialis Powder, Haematoxylon Campechianum Wood Extract, Henna, Iron Oxides, Katsu201, Ki4, Ki5, Ki201, Ki202(1), Ki202(2), Ki203, Ki204, Ki205, Ki401, Ki402, Ki403(1), Ki404, Ki405, Ki406, Ki407, Kuro401, Lactoflavin, Lawsone, Magnesium Stearate, Manganesa Violet, Mica, Midori3, Midori201, Midori202, Midori204, k Midori205, Midori401, Midori402, Murasaki201, Murasaki401, Natural Red 26, Ninhydrin, Orange 4, Orange 4 Lake, Orange 5, Orange 5 Lake, Orange 10, Orange 10 Lake, Orange 11, Oxobenzoxazinyl Naphthalene Sulfoanilide, Pigment Blue 15:2, Pigment Green 7, Pigment Orange 5, Pigment Red 4, Pigment Red 5, Pigment Red 48, Pigment Red 53, Pigment Red 53:1, Pigment Red 57, Pigment Red 57:1, Pigment Red 63:1, Pigment Red 64:1, Pigment Red 68, Pigment Red 83, Pigment Red 88, Pigment Red 90:1 Aluminum Lake, Pigment Red 112, Pigment Red 172 Aluminum Lake, Pigment Red 173 Aluminum Lake, Pigment Red 190, Pigment Violet 19, Pigment Yellow 1, Pigment Yellow 3, Pigment Yellow 12, Pigment Yellow 73, Ponceau SX, Pyrophyllite, Red 4, Red 4 Lake, Red 6, Red 6 Lake, Red 7, Red 7 Lake, Red 17, Red 21, Red 21 Lake, Red 22, Red 22 Lake, Red 27, Red 27 Lake, Red 28, Red 28 Lake, Red 30, Red 30 Lake, Red 31, Red 31 Lake, Red 33, Red 33 Lake, Red 34, Red 34 Lake, Red 36, Red 36 Lake, Red 40, Red 40 Lake, Silver, Sodium 5-Nitroguaiacolate, Sodium Zinc Cetyl Phosphate, Solvent Green 3, Solvent Green 7, Solvent Orange 1, Solvent Red 1, Solvent Red 3, Solvent Red 23, Solvent Red 24, Solvent Red 43, Solvent Red 48, Solvent Red 49:1, Solvent Red 72, Solvent Red 73, Solvent Violet 13, Solvent Yellow 18, Solvent Yellow 29, Solvent Yellow 33, Solvent Yellow 44, Sunset Yellow, Sunset Yellow Aluminum Lake, Titanium Dioxide, Titanium Oxynitride, Titanium/Titanium Dioxide, Ultramarines, Umber, Vat Red 1, Violet 2, Yellow 5, Yellow 5 Lake, Yellow 6, Yellow 6 Lake, Yellow 7, Yellow 7 Lake, Yellow 8, Yellow 10, Yellow 10 Lake, Yellow 11, Yellow Ocher, Zinc Oxide, Zinc Stearate.

In other aspects, the invention is directed to compositions containing at least one of each of the three agents discussed above and photosensitive compounds such as polyunsaturated color and aromatic compounds, dyes, organic pigments, antioxidants, fragrance compounds or flavor compounds. These compositions can be in the form of a complete retail cosmetic, personal care product, or household products or an intermediate to a complete retail product. Therefore, the compositions of this invention may optionally contain one or more carriers or solubilizers used in cosmetics, personal care or household products, for example, esters of long-chain fatty acids or short or long chain alcohols.

Amounts of the total combination of three agents typically range from 0.1 to 40 wt % based on the total weight of the composition. More typically, the amount falls within the range of 1 wt % to 25 wt %, even more preferably ranges from about 2 wt % to about 15 wt %. Typical ratios of stabilizer to color, or antioxidant, or flavor or fragrances are 1:1 to 1:10, preferably between 1:1 to 1:5.

Additional ingredients that can be used in the compositions of this invention are organic sunscreen agents, metal oxide sunscreen agents, dispersing agents, preservatives, anti-foams, perfumes, oils, waxes, propellants, dyes, pigment emulsifiers, surfactants, thickeners, humectants, exfoliants and emollients. These compositions may be in the form of a cosmetic composition with a cosmetically acceptable carrier and one or more cosmetic adjuvants. The compositions can optionally have antioxidants or other stabilizers which do not have UV absorbing characteristics.

The methods of using the complete retail products that are the compositions of the invention comprise applying said composition to a substrate, for example, skin, hair, leather, fabrics, for example, clothing articles, wigs, etc. Preferred substrates are skin and hair.

The compositions of this invention may contain dispersing agents, emulsifiers or thickening agents to assist in applying a uniform layer of the active compounds. Suitable dispersing agents for the composition include those useful for dispersing organic or inorganic sunscreen agents in either a water phase, oil phase, or part of an emulsion, including, for example, chitosan.

Emulsifiers may be used in the compositions of this invention, for example, sunscreen formulations, to disperse one or more of the components in the combinations. Suitable emulsifiers include agents such as, for example, glycerol stearate, stearyl alcohol, cetyl alcohol, dimethicone copolyol phosphate, hexadecyl-D-glucoside, octadecyl-D-glucoside, etc.

Thickening agents may be used to increase the viscosity of the compositions. Suitable thickening agents include carbomers, acrylate/acrylonitrile copolymers, xanthan gum and combinations of these. The carbomer thickeners include the crosslinked CARBOPOL® acrylic polymers from B.F. Goodrich. The amount of thickener within the composition, for example, a sunscreen formulation, on a solids basis without water, may range from about 0.001 to about 5%, preferably from 0.01 to about 1% and optimally from about 0.1 to about 0.5% by weight.

Minor optional adjunct ingredients for the compositions may include preservatives, waterproofing agents, fragrances, anti-foam agents, plant extracts (Aloe vera, witch hazel, cucumber, etc) opacifiers, skin conditioning agents and colorants, each in amounts effective to accomplish their respective functions.

The compositions may optionally contain an ingredient which enhances the waterproof properties such as, compounds that form a polymeric film, such as dimethicone copolyol phosphate, diisostearoyl trimethyolpropane siloxysilicate, chitosan, dimethicone, polyethylene, polyvinylpyrrolidone (PVP), polyvinylpyrrolidone/vinylacetate, PVP/Eiconsene copolymer and adipic acids/diethylene glycol/glycerine crosspolymer etc. Waterproofing agents may be present at levels of from about 0.01 to about 10% by weight.

The compositions, may also optionally contain one or more skin conditioning agents. These include humectants, exfoliants and emollients.

Humectants are polyhydric alcohols intended for moisturizing, reducing scaling and stimulating the removal of built scale from the skin. Typically polyhydric alcohols include polyalkylene glycols and more preferably alkylene polyols and their derivatives. Illustrative are propylene glycol, dipropylene glycol, polypropylene glycol, polyethylene glycol, sorbitol, 2-pyrrolidone-5-carboxylate, hydroxypropyl sorbitol, hexylene glycol, ethoxydiglycol 1,3-butylene glycol, 1,2,6-hexanetriol, glycerin, ethoxylated glycerin, propoxylated glycerin and mixtures thereof. Most preferably the humectant is glycerin. Amounts of humectant can range anywhere from 1 to 30%, preferably from 2 to 20% and optimally from about 5 to 10% by weight of the composition.

The exfoliants suitable for use in the present may be selected from alpha-hydroxy carboxylic acids, beta hydroxycarboxylic acids and salts of these acids. Most preferred are glycolic, lactic and salicylic acids and their alkali, metal or ammonium salts.

Suitable emollients include those agents known for softening the skin or hair which may be selected from hydrocarbons, fatty acids, fatty alcohols and esters. Petrolatum is a common hydrocarbon type of emollient conditioning agent. Other hydrocarbons that may be employed include alkyl benzoate, mineral oil, polyolefins such as polydecene, and paraffins, such as isohexadecane. Fatty acids and alcohols typically have from about 10 to 30 carbon atoms. Illustrative are myristic, isostearic, hydroxystearic, oleic, linoleic, ricinoleic, behenic and eruicic acids and alcohols. Oily ester emollients may be those selected from one or more of the following, triglyceride esters, acetoglyceride esters, ethoxylated glycerides, alkyl esters of fatty acids, ether esters, polyhydric alcohol esters and wax esters. Additional emollients or hydrophobic agents include C₁₂ to C₁₅ alkyl benzoate, dioctyladipate, octyl stearate, octyldodecanol, hexyl laurate, octyldodecyl neopentanoate, cyclomethicone, dicapryl ether, dimethicone, phenyl trimethicone, isopropyl myristate, capriylic/capric glycerides, propylene glycol dicaprylate/dicaprate and decyl oleate, isopropyl citrate, diisopropyl adipate, ethylhexyl neopentanoate, isopropyl laurate, hexyl laurate, C₁₂₋₁₅ alkyl benzoate, ethylhexyl palmitate, octyldodecyl neopentatnoate, ethylhexyl state etc.

Examples of esters of long-chain fatty acids, for example, include: long-chain fatty acid esters of retinol; long-chain fatty acid ester of ascorbic acid; long-chain fatty acid ester of glycerol; etc. The long-chain fatty acid ester of retinol can be selected from the group consisting of retinyl palmitate, retinyl myristate, and retinyl stearate. Most preferably, the retinyl ester is retinyl palmitate. Preferably, the retinyl ester comprises from about 0.05% to about 0.15% of a skin cream composition. The long-chain fatty acid ester of ascorbate can be selected from the group consisting of ascorbyl myristate, ascorbyl palmitate, and ascorbyl stearate. Preferably, the ascorbic acid ester is ascorbyl palmitate. Preferably, the ascorbic acid ester comprises from about 0.01% to about 0.02 to 0.03% of the composition.

The long-chain fatty acid ester of glycerol can be selected from the group consisting of glyceryl stearate, glyceryl palmitate, and glyceryl arachidate. Preferably, the ester of glycerol is glyceryl stearate. Preferably, the glyceryl stearate comprises from 0.5% to about 0.7% of the composition.

Examples of short or long chain alcohols, for example, include: hexyl (chain-length, C-6), caprylyl (C-8), decyl (C-10), lauryl (C-12), myristyl (C-14), cetyl (C-16), stearyl (C-18), arachidyl (C-20), behenyl alcohols (C-22).

The compositions may optionally contain one or more inorganic sunscreen agents as discussed above including micro fine surface treated titanium dioxide and micro fine untreated and surface treated zinc oxide. Titanium dioxide in the compositions preferably has a mean primary particle size of between 5 and 150 nm and preferably from 10 to 100 nm. Titanium oxide may have anatase, rutile or amorphous structure. The zinc oxide in the sunscreen compositions preferably has a mean primary particle size of between 5 nm and 150 nm, preferably between 10 nm and 100 nm. Examples of modified titanium dioxide compositions include:

Eusolex® T-45D (surface treated with alumina and simethicone, 45% dispersion in isononoyl isononoate);

Eusolex®T-Aqua, (surface treated with aluminum hydroxide, 25% dispersion in water); and

Eusolex® T-2000 (surface treated with alumina and simethicone),

Eusolex® TS (surface treated with aluminum stearate), and

Eusolex® T-AVO (surface treated with silica), all available from MERCK KGaA.

Independent of the organic sunscreens which form part of the combination of agents, particularly useful organic sunscreen agents that can be introduced are Avobenzone, 2-ethylhexyl p-methoxycinnamate, 4,4′-t-butylmethoxydibenzoyl methane, 2 hydroxy-4-methoxybenzophenone, octyldimethyl p-aminobenzoic acid, 2,2-dihydroxy-4-methoxybenzophenone, ethyl-4-[bis(hydroxypropyl)]aminobenzoate, 2-ethylhexyl-2-cyano-3,3-diphenylacrylate, 2-ethylhexylsalicylate, glycerol p-aminobenzoate, 3,3,5-trimethylcyclohexylsalicylate, methylanthranilate, p-dimethylaminobenzoic acid, 2-ethylhexyl p-dimethylaminobenzoate, 2-phenylbenzimidazole-5-5-sulfonic acid, 2-(p-dimethylamino phenyl-5-sulfoniobenzoxazoic acid and mixtures thereof.

Examples of useful commercially available organic sunscreen agents that can be introduced include 2-phenylbenzimidazole-5-sulphonic acid, 2-(4-methylbenzylidene)-camphor, 4-isopropyldibenzoyl methane all of the Eusolex™ series sold by EMD Chemicals and Merck KGaA, Darmstadt, Germany.

The compositions may also contain one or more additional monomeric organic chromophoric compounds. These can either be UV-A, UV-B or broad band filters. Examples of suitable UV-A sunscreens include benzophenone derivatives, menthyl anthranilate, butyl methoxydibenzoyl methane and benzylidene-dioxoimidazoline derivatives. Examples of suitable UV-B sunscreens include cinnamate derivatives, salicylate derivatives, para-aminobenzoic acid derivatives, camphor derivatives, phenylbenzimidazole derivatives and diphenylacrylate derivatives. Examples of suitable broad-band sunscreen include benzotriazole derivatives and triazine derivatives such as anisotriazone. Others include ethylhexyltriazone and diethylhexylbutamidotriazone.

The compositions may contain a conventional antioxidant. Examples of suitable antioxidants which provide stability include p-hydroxybenzoic acid and its derivatives (ethylisobutyl, glyceryl esters of p-hydroxybenzoic acid gallic acid, ellagic acid, ascorbic acid,); salicylates (octylamyl, phenyl, benzyl menthyl, glycerol and dipropyleneglycol esters); butyl hydroxy toluene (BHT), butyl hydroxy anisole (BHA).

Cumarin derivatives; flavones; hydroxy or methoxy substituted benzophenones; uric or tannic acid and its derivatives; and benzophenones. Also, the compositions may contain natural antioxidants, such as, Emblica antioxidant, Pine antioxidant, Grape antioxidant, rosem, aryantioxidate, Green tea antioxidant and others. The combination of three agents described above can be used to protect conventional antioxidants from photodegradation in personal care formulations such as hair care, skin care and cosmetic formulations.

The entire disclosure of all applications, patents and publications, cited above are hereby incorporated by reference. All percentages are by weight.

### EXPERIMENTAL

### Synergistic blend of Color Stabilizer

### Materials and Method:

### Part I

A) Stabilization of water-soluble dyes in Surfactant Standard Formulation

Different dyes (Table 1) were investigated in surfactant based system (Table 2) using different photostabilizers (Table 3). All samples were filled into 30 ml transparent borax glass bottles. One part of each formula was stored in the dark condition as a control (Un-irradiated). The other samples were irradiated by Sun Simulator (SUNTEST CPS⁺, ATLAS Material Testing Solutions, Inc.) in assigned time intervals under the following conditions: 1) Using solar standard filter, 2) Irradiance Energy (300-800 nm)=550 W/m², 3) Measured UV dose (290-400 nm)=83 W/m² 4) 1 hour with 83 W/m² (290-400 nm)˜300 kJ/m², 5) Irradiation time=up to 24 hours, 6) Temperature of sample chamber=35-40° C. The results of this study are illustrated in FIGS. 1 & 2.

B) Stabilization of water-soluble dyes in Surfactant Commercial Product (Shampoo)

Different commercially available shampoos (Table 4) were tested using different stabilizers (Table 5). Test Conditions were identical with Part I (A). The results are illustrated in FIGS. 3 & 4.

### Part II

### Stabilization of Water-Soluble Dyes in Highly Fragranced Standard Formulation

Different dyes (Table 6) were investigated in highly fragranced water-alcohol formulation (Table 7) using different photostabilizers (Table 3). All samples were filled into 30 ml transparent borax glass bottles. One part of each formula was stored in the dark condition as a control. The other samples were irradiated by Sun Simulator (SUNTEST CPS⁺, ATLAS Material Testing Solutions, Inc.) in assigned time intervals under the following conditions: 1) Using solar standard filter, 2) Irradiance Energy (300-800 nm)=550 W/m², 3) Measured UV dose (290-400 nm)=83 W/m² 4) 1 hour with 83 W/m² (290-400 nm)˜300 kJ/m², 5) Irradiation time=up to 24 hours, 6) Temperature of sample chamber=35-40° C. The results of this study are illustrated in FIGS. 5 & 6.

### B) Stabilization of Water-Soluble Dyes in Highly Fragranced Commercial Product (Body Mist)

Two commercial highly fragranced products (Body Mist Type) (Table 8) were tested using two different stabilizers (Table 5). Test Conditions were identical with Part II (A). The results are illustrated in FIGS. 7 & 8.

TABLE 1
Water Soluble dyes (Used in Surfactant Standard Formulation)

| Colorants | Color Index | Amount (% w/w) |
|---|---|---|
| D&C Red#33 | CI 17200 | 0.10 |
| FD&C Red#40 | CI 16035 | 0.10 |
| Ext.Violet#2 | CI 60730 | 0.06 |

TABLE 2
Surfactant Standard Formulation

| INCI Name | Trade Name/Supplier | % w/w |
|---|---|---|
| Phase A | | |
| Water (demineralized) | | qs. |
| Disodium EDTA | Triplex III/Rona | 0.10 |
| Sodium Laureth Sulfate | Standapol ES-2/Cognis | 40.00 |
| (25% actives) | | |
| Cocamidopropyl Betaine | Velvetex BA-35/Cognis or | 10.00 |
| (30% actives) | Mackam 35-HP/MacIntyre | |
| Propylene glycol, DMDM | Paragon/McIntyre | 1.00 |
| hydantoin, Methylparaben | | |
| Phase B | | |
| Photostabilizers | See Table 3 | 0.00-0.10 |
| PEG-40 Hydrogenated Castor | Universal Preserv-A-Chem | 2.00 |
| Oil | | |
| Phase C | | |
| Citric acid (10% aqueous | To adjust pH to 5.5-5.7 | 0.30-0.40 |
| solution) | | |
| Colorants (1% aqueous solution) | See Table 1 | 0.06-0.10 |
| Total | | 100 |

TABLE 3
Photostabilizers (Used in Standard Formulation)

| Trade name | INCI Name |
|---|---|
| Oxynex ST | Diethylhexyl Syringylidene Malonate, |
| Liquid | Caprylic/Capric Triglyceride |
| Oxynex ST | Diethylhexyl Syringylidene Malonate, |
| Blend | Caprylic/Capric Triglyceride, Homosalate, Butyl |
| ST:HMS:Avo | Methoxydibenzoylmethane |
| (10:80:10) | |
| Oxynex ST | Diethylhexyl Syringylidene Malonate, |
| Blend | Caprylic/Capric Triglyceride, Ethylhexyl |
| ST:OMC:Avo | Methoxycinnamate, Butyl |
| (10:80:10) | Methoxydibenzoylmethane |
| Oxynex ST | Diethylhexyl Syringylidene Malonate, |
| Blend | Caprylic/Capric Triglyceride, Ethylhexyl |
| ST:OMC:Avo | Methoxycinnamate, Butyl |
| (15:70:15) | Methoxydibenzoylmethane |
| Oxynex ST | Diethylhexyl Syringylidene Malonate, |
| Blend | Caprylic/Capric Triglyceride, Ethylhexyl |
| ST:OMC:Avo | Methoxycinnamate, Butyl |
| (10:70:20) | Methoxydibenzoylmethane |

TABLE 4
Surfactant-Based Commercial Products

| Trade Name | Type | Dye Ingredients | Company |
|---|---|---|---|
| Suave ® Naturals ® | Shampoo | D&C Red No. 33 | Unilever |
| (Fresh Mountain | | (CI 17200), | |
| Strawberry) | | D&C Orange | |
| | | No. 4 (CI 15510) | |
| Suave ® Naturals ® | Shampoo | Ext. Violet 2 | Unilever |
| (Lavender & Lilac) | | (CI 60730) | |
| Alberto VO5 Extra Body | Shampoo | Blue 1, Yellow 5, | Alberto- |
| | | Red 40 | Culver USA |

TABLE 5
Photostabilizers (Used in Commercial Products)

| Trade name | INCI Name |
|---|---|
| Oxynex ST | Diethylhexyl Syringylidene Malonate, |
| Liquid | Caprylic/Capric Triglyceride |
| Oxynex ST | Diethylhexyl Syringylidene Malonate, |
| Blend | Caprylic/Capric Triglyceride, Ethylhexyl |
| ST:OMC:Avo | Methoxycinnamate, Butyl |
| (10:80:10) | Methoxydibenzoylmethane |

TABLE 6
Water Soluble dyes (Used in Highly Fragranced Standard Formulation)

| Colorants | Color Index | Amount (% w/w) |
|---|---|---|
| D&C Red No. 33 | CI 17200 | 0.10 |
| Ext. D&C Violet | CI 60730 | 0.10 |
| No. 2 | | |

TABLE 7
Highly Fragranced Standard Formulation

| INCI Name | Trade Name/Supplier | % w/w |
|---|---|---|
| Water | Water (demineralized) | qs |
| Alcohol, Reagent | OmniSolv/EMD | 65.00 |
| Water-soluble dyes | See Table 6 | 0.10 |
| (1% aqueous | | |
| solution) | | |
| Fragrance | VOGUE # FRAG 230934/ | 4.00 |
| | drom fragrances | |
| | international | |
| Photostabilizers | See Table 3 | 0.00-0.50 |
| Total | | 100 |

TABLE 8
Highly Fragranced Commercial Products

| Trade Name | Type | Dye Ingredients | Company |
|---|---|---|---|
| CALGON | Body | Ext.D&C Violet No. 2, D&C Red | COTY |
| tropical dream | Mist | No. 33 | |
| CALGON | Body | Ext.D&C Violet No. 2, D&C Red | COTY |
| morning glory | Mist | No. 33, FD&C Blue No. 1 | |

### BRIEF DESCRIPTION OF THE DRAWINGS

FIGS. 1-8 are photographs of sample jars with dyed formulations.

FIG. 1 shows Red No. 33 in Surfactant Standard Formulation;

FIG. 2 shows Ext. D&C Violet No. 2 in Surfactant Standard Formulation;

FIG. 3 shows D&C Red No. 33, D&C Orange No. 4 in Suave® Naturals® (Fresh Mountain Strawberry) Shampoo;

FIG. 4 shows Ext. D&C Violent No. 2 in Suave® Naturals® (Lavender & Lilac) Shampoo;

FIG. 5 shows Red No. 33 in Highly Fragranced Standard Formulation;

FIG. 6 shows Ext. D&C Violet No. 2 in Highly Fragranced Standard Formulation;

FIG. 7 shows Ext. D&C Violet No. 2, D&C Red No. 33 in CALGON tropical dream Body Mist, and

FIG. 8 shows Ext. D&C Violet No. 2, D&C Red No. 33, FD&C Blue No. 1 in CALGON morning glory Body Mist.

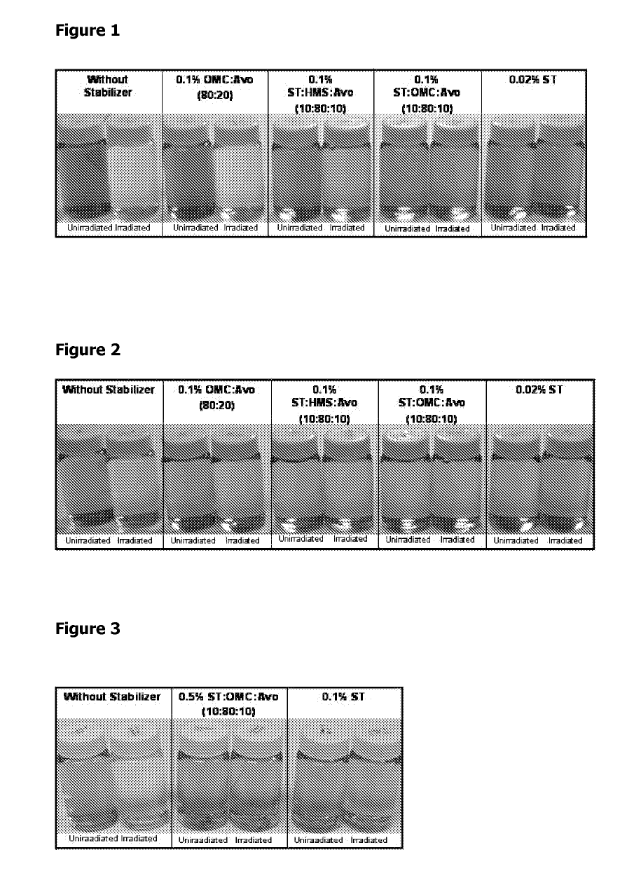

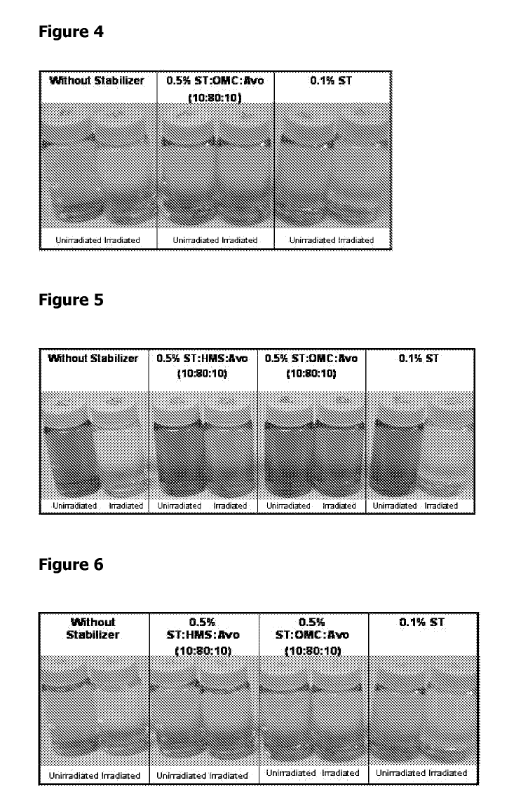

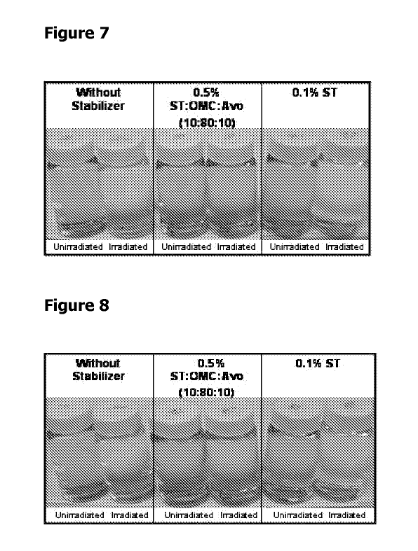

### CONCLUSION

A composition comprising a stabilizer belonging to the Benzylidene malonate group of products when combined with UV-A organic sunscreens, such as, dibenzoyl methane class of UV-A filters and UV-B organic sunscreens, such as Octylmethoxy cinnamate, Octyl salicylate, homomethylsalicylate gives a superior color and fragrance stabilizing effects. Combination benzylidene malonate with other UB-A and UV-A sunscreens can also be envisioned as superior color stabilizer.

Prophetic Formulations of Typical Exemplary Cosmetic, Personal Care & Household Formulations where Stabilizer(s) of the present Invention can be included.

All combinations are of Diethylhexyl syringylidene malonate (Oxynex®ST, methoxydibenzoylmethane and Homosalate at a ratio of 10:80:10

Formulation 1. Shower Gel

Stabilization of D&C Color Red #33

| Water | qs to 100 |
|---|---|
| Cocoamidopropyl betaine | 5.00 |
| Sodium laureth sulfate | 5.50 |
| Sodium lauryl sarcosinate | 0.50 |
| Polyquaternium-10 | 0.10 |
| Sodium sulfate | 2.10 |
| Polysorbate 2 | 1.00 |
| PEG-40 hydrogenated castor oil | 0.50 |
| Combination of photostabilizers | 0.50 |
| D&C Red #33 | 0.05 |
| DMDM Hydantoin | 0.60 |

Formulation 2. Moisturizing Cleansing Gel

Stabilization of FD&C Blue #1

| Water | qs to 100 |
|---|---|
| Sodium laureth sulfate | 16.00 |
| Lanolin alcohol | 2.50 |
| Cocamidopropyl betaine | 2.00 |
| Stearic acid | 1.25 |
| Glycereth-12 | 1.00 |
| Polyquaternium-10 | 0.25 |
| Glycerin | 5.00 |
| Combination of photostabilizers | 0.50 |
| FD&C Blue #1 | 0.01 |
| Phenoxyethanol | 0.60 |
| Methylparaben | 0.20 |
| Propylparaben | 0.10 |

Formulation 4. Pearlescent Conditioning Shampoo

Stabilization of Color and Viscosity

| Water | qs to 100 |
|---|---|
| Guar hydroxyproyltrimonium | 0.5 |
| Chloride | |
| Hydroxypropyl methylcellulose | 0.60 |
| TEA-lauryl sulfate | 7.00 |
| Glycol stearate | 1.20 |
| Sodium lauroamphoacetate | 5.00 |
| Sodium trideceth sulfate | 3.00 |
| Cocamide DEA | 2.50 |
| Combination of photostabilizers | 0.50 |
| D&C Red #33 | 0.04 |
| FD&C Blue #1 | 0.01 |
| DMDM Hydantoin | 0.60 |

Formulation 5. Baby Shampoo

Stabilization of Viscosity

| Water | qs to 100 |
|---|---|
| PEG-80 sorbitan laurate | 12.00 |
| Sodium trideceth sulfate | 5.00 |
| Sodium lauroamphoacetate | 5.00 |
| PEG-120 methyl glucose dioleate | 2.00 |
| Cocamidopropyl hydroxysultaine | 1.00 |
| Polysorbate 20 | 0.80 |
| Combination of photostabilizers | 0.10 |
| Guaiazulene | 0.010 |
| DMDM Hydantoin | 0.50 |

Formulation 6. Rinse off Conditioner

Stabilization of Color and Fragrance

| Water | qs to 100 |
|---|---|
| Glycerin | 2.00 |
| Disodium EDTA | 0.10 |
| Steramidopropyl dimethylamine lactate | 2.00 |
| Cetyl alcohol | 2.50 |
| Stearyl alcohol | 2.00 |
| Dimethicone | 2.50 |
| Cyclomethicone | 3.00 |
| Lactic acid | 0.50 |
| Combination of photostabilizers | 1.00 |
| 3-cyclohexene-1-carboxaldehyde, 2,4-dimethyl | 0.40 |
| D&C Green #3 | 0.01 |

Formulation 8. Hair Styling Gel

Stabilization of Polymer and Viscosity

| Water | qs |
|---|---|
| Carbomer | 0.50 |
| Glycerin | 1.00 |
| Panthenol | 0.10 |
| Disodium EDTA | 0.05 |
| PVP K-30 | 2.00 |
| Combination of photostabilizers | 0.50 |
| Polysorbate-20 | 1.00 |
| Peg-40 Hydrogenated Castor oil | 0.50 |
| FD&C Blue#1 | 0.01 |
| Aminomethylpropanol | 0.50 |
| Diazolidinyl urea | 0.30 |

Formulation 10. Temporary Hair Color

Stabilization of Color and Viscosity

| | |
|---|---|
| Propylene Glycol | 3.00 |
| Hydroxyethylcellulose | 1.00 |
| Sodium hydroxide | 0.04 |
| Methyl paraben | 0.20 |
| DMDM hydantoin | 0.50 |
| Combination of photostabilizers | 0.50 |
| Polysorbate 20 | 1.00 |
| FD&C Blue#1 | 0.04 |
| D&C Orange #4 | 0.20 |
| D&C Yellow #10 | 0.03 |
| D&C Green #5 | 0.02 |
| FD&C Red #4 | 0.05 |
| Water | qs |

Formulation 11. O/W Lotion

Stabilization of Color

| Water | qs |
|---|---|
| Glycerin | 5.00 |
| Disodium ETDA | 0.10 |
| Xantham Gum | 0.20 |
| Isopropyl Palmitate | 5.00 |
| Caprylic/Capric triglycerides | 3.00 |
| Apricot Kernel oil | 3.00 |
| Glycerol stearate | 2.50 |
| Peg-100 stearate | 1.50 |
| Dimethicone | 1.00 |
| Stearic acid | 1.50 |
| Triethanolamine | 0.10 |
| Phenoxyethanol | 0.60 |
| Methylparaben | 0.20 |
| Propylparaben | 0.10 |
| Combination of photostabilizers | 0.10 |
| D&C Green# 3 | 0.01 |
| Fragrance | 0.10 |

Formulation 12. Self Tanner Foam

Stabilization of Color

| Water | qs |
|---|---|
| Cetyl hydroxyethylcellulose | 0.25 |
| Sorbitol | 2.50 |
| Propylene Glycol | 5.00 |
| Dihydroxyacetone | 6.00 |
| Decyl Glucoside | 1.50 |
| Polysorbate 20 | 1.00 |
| Combination of photostabilizers | 0.50 |
| Carmine | 0.30 |
| FD&C Yellow #5 | 0.05 |
| FD&C Blue#1 | 0.02 |
| Caramel | 0.50 |
| Citric Acid | 0.04 |
| Phenoxyethanol | 0.50 |
| Methylparaben | 0.20 |
| Propylparaben | 0.10 |
| Fragrance | 0.10 |

Formulation 13. Skin Toner

Stabilization of Color

| Water | qs |
|---|---|
| SD alcohol 40B | 15.00 |
| Glycerin | 5.00 |
| Aloe vera gel | 3.00 |
| Polysorbate 20 | 1.00 |
| Peg-40 hydrogenated castor oil | 0.50 |
| Combination of photostabilizers | 0.20 |
| FD&C blue #1 | 0.01 |
| D&C Red #33 | 0.01 |
| Methylparaben | 0.20 |
| DMDM hydantoin | 0.50 |
| Fragrance | 0.10 |

Formulation 14. Facial Cleanser

Stabilization of Color and Viscosity

| | |
|---|---|
| Water | 60.00 |
| Propylene glycol | 1.00 |
| Peg-150 pentaerythrityl tetrastearate | 1.50 |
| Dimethicone copolyol | 5.00 |
| Disodium cocoamphoacetate | 3.50 |
| Sodium lauroyl sarcosinate | 3.50 |
| Citric acid | qs to pH-6 |
| Combination of photostabilizers | 0.20 |
| Chlorophyll copper | 0.01 |
| DMDm Hydantoin | 0.60 |

Formulation 15. Clear Deodorant

Stabilization of Fragrance

| | |
|---|---|
| Water | 5.00 |
| Propylene glycol | 46.75 |
| Sodium Stearate | 8.00 |
| PPG-3 myristyl ether | 39.00 |
| Triclosan | 0.25 |
| Combination of photostabilizers | 2.00 |
| FD&C Blue #1 | 0.01 |
| 3-cyclohexene-1-carboxaldehyde,1-methyl- | 0.5% |
| 4-(4-methyl-3-pentenyl) | |

Formulation 16. Clear Gel Toothpaste

Stabilization of Color & Maintenance of Product Integrity

| Water | qs to 100 |
|---|---|
| Glycerin | 10.00 |
| Sorbitol(70%) | 30.00 |
| PEG-32 | 5.00 |
| Hydrated silica abrasive | 15.00 |
| Hydrated silica thickener | 8.00 |
| Sodium sachharin | 0.30 |
| Sodium monofluoro phosphate | 0.80 |
| Sodium lauryl sulfate | 1.30 |
| Combination of photostabilizers | 0.50 |
| Polysorbate 20 | 1.00 |
| FD&C Blue # 1 | 0.05 |
| Flavor | 1.00 |

Formulation 17. Hydroalcoholic Mouthwash

Stabilization of Color

| | |
|---|---|
| Water | 75.18 |
| Glycerin | 8.00 |
| Sodium benzoate | 0.10 |
| Benzoic acid | 0.04 |
| Sodium saccharin | 0.08 |
| Cetylpyridinium chloride | 0.05 |
| FD&C Blue #1 | 0.02 |
| SDA alcohol 40B | 15.00 |
| Polysorbate20 | 1.00 |
| Combination of photostabilizers | 0.50 |

Formulation 18. Insect Repellent lotion

Stabilization of Color and Insect Repellant

| Water | qs to 100 |
|---|---|
| Glycerin | 5.00 |
| Disodium EDTA | 0.10 |
| Xanthan Gum | 0.25 |
| Ethyl Butylaminopropionate | 10.00 |
| PPG-3 Benzyl Ether Myristate | 2.50 |
| Isopropyl myristate | 3.00 |
| Glyceryl stearate | 2.00 |
| PEG-100 stearate | 1.00 |
| Stearic acid | 1.00 |
| Cetyl alcohol | 1.50 |
| Phenoxyethanol | 0.60 |
| Methylparaben | 0.20 |
| Propylparaben | 0.10 |
| Combination of photostabilizers | 0.50 |
| FD&C Blue #1 | 0.05 |
| Fragrance | 0.10 |

Formulation 19. Lipstick

Stabilization of Color

| | |
|---|---|
| Castor oil | 7.70 |
| Candelilla wax | 2.30 |
| Carnauba wax | 2.00 |
| Ozokerite | 2.00 |
| Microcrystalline wax | 4.00 |
| Caprylic/capric triglycerides | 25.00 |
| Octyldodecanol | 6.00 |
| Methylparaben | 0.20 |
| Propylparaben | 0.10 |
| Diethylhexyl syringylidene malonate | 1.00 |
| D&C Red #27 | 0.10 |
| FD&C Red # 21 | 0.20 |
| FD&C Yellow #5 Aluminum Lake | 0.10 |

Formulation 20. Lipgloss

Maintenance of Product Integrity

| | |
|---|---|
| Polyisobutene | 30.00 |
| Petrolatum | 25.00 |
| Ozokerite | 5.00 |
| Microcrystalline wax | 1.00 |
| Octyldodecanol | 10.00 |
| Castor oil | qs to 100 |
| Diethylhexyl syringylidene malonate | 0.50 |
| D&C Red # 22 aluminum lake | 0.05 |
| Titanium dioxide | 0.10 |
| Methylparaben | 0.20 |
| Propylparaben | 0.10 |

Formulation 21. Nail Lacquer

Stabilization of Color

| | |
|---|---|
| Butyl acetate | 42.00 |
| Ethyl acetate | 18.10 |
| Stearalkonium hectorite | 1.20 |
| Dibutyl phthalate | 5.00 |
| Tosylamide/formaldehyde resin | 7.00 |
| Isopropanol | 8.00 |
| Nitrocellulose | 4.00 |
| Combination of photostabilizer | |
| D&C Red # 27 Aluminum lake | 0.10 |
| D&C Red# 34 Calcium lake | 0.10 |

Formulation 22. Dishwashing Liquid

Stabilization of Color and Maintenance of Product Integrity

| | |
|---|---|
| Water | 42.00 |
| Carbomer | 1.00 |
| Ammonium lauryl sulfate | 7.00 |
| Sodium laureth sulfate | 7.50 |
| Combination of photostabilizers | 0.50 |
| Polysorbate 20 | 1.00 |
| D&C Red #33 | 0.01 |
| FD&C Blue #1 | 0.01 |
| Sodium citrate | 0.50 |
| Sodium hydroxide | 0.90 |

Formulation 23. Liquid Laundry Detergent

Stabilization of Color and Fragrance

| Water | qs to 100 |
|---|---|
| Sodium lauryl sulfate | 7.50 |
| Stearic Acid | 3.00 |
| Palmitic Acid | 3.50 |
| C14-15 Pareth-7 | 17.00 |
| Triethanolamine | 7.50 |
| Propylene glycol | 12.00 |
| Citric acid | 3.25 |
| Potassium hydroxide | 4.25 |
| Combination of photostabilizers | 4.00 |
| FD&C Blue # 1 | 0.01 |
| 2-ethyl hexanal cycloglycol acetate | 1.00 |

As used herein and in the claims, when “a” compound or class of compounds is named, or “at least a” or “at least one” compound or class of compounds is named, it is understood that “one or more” is meant and intended, for example, from each class of compounds named.

The preceding examples can be repeated with similar success by substituting the generically or specifically described reactants and/or operating conditions of this invention for those used in the examples.

The entire disclosures of all applications, patents and publications, cited herein and of corresponding U.S. Provisional Application Ser. No. 60/847, 911, filed Sep. 29, 2007, are incorporated by reference herein.

From the foregoing description, one skilled in the art can easily ascertain the essential characteristics of this invention, and without departing from the spirit and scope thereof, can make various changes and modification of the invention to adapt it to various usages and conditions.

What is claimed is:

1. A method of stabilizing a photosensitive compound comprising combining three photostabilizing agents with said photosensitive compound in a ratio within a range of 10:1 to 1:2 based on total weight percent of the three photostabilizing agents to the total weight of the photosensitive compound, wherein said three photostabilizing agents comprise
A) at least one dialkylbenzalmalonate;
B) at least one UV-A organic sunscreen, and
C) at least one UV-B organic sunscreen.

2. A method as in claim 1 wherein the dialkylbenzalmalonate is of the formula I or II 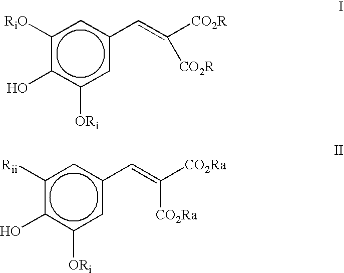
wherein, each Rᵢ is independently linear or branched C₁-C₈ alkyl and each R is independently linear or branched C₁ to C₂₀ alkyl, Rᵢᵢ is hydrogen or linear or branched C₁-C₈ alkyl and each Ra is independently, linear or branched C₁ to C₃₀ alkyl.

3. A method as in claim 1 wherein for the dialkylbenzalmalonate of the formula I and II,
each Rᵢ is independently linear or branched C₁-C₄ alkyl and each R is independently linear or branched C₁-C₁₂ alkyl.

4. A method as in claim 1 wherein the dialkylbenzalmalonate is of one of the formulae III, IV, V and VI 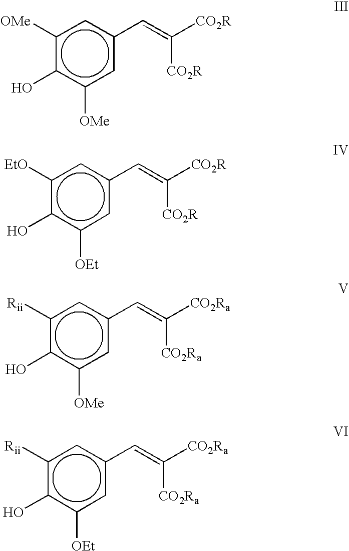
wherein
R is linear or branched C₁ to C₂₀ alkyl,
Rₐ is linear or branched C₁ to C₃₀ alkyl, and
Rᵢᵢ is linear or branched C₁-C₈ alkyl or hydrogen.

5. A method as in claim 1 wherein the dialkylbenzalmalonate derivative is one or more of
diethyl-3,5-dimethoxy-4-hydroxy benzylidene malonate,
di-(2-ethylhexyl)-3,5-dimethoxy-4-hydroxy benzylidene malonate,
diisoamyl-3,5-dimethoxy-4-hydroxy benzylidene malonate,
didodecyl-3,5-dimethoxy-4-hydroxy benzylidene malonate,
dipalmitoyl-3,5-dimethoxy-4-hydroxy benzylidene malonate,
di-isopropyl-3,5-dimethoxy-4-hydroxy benzylidene malonate,
diethyl-3-methoxy-4-hydroxy benzylidene malonate,
di-(2-ethylhexyl)-3-methoxy-4-hydroxy benzylidene malonate,
diisoamyl-3-methoxy-4-hydroxy benzylidene malonate,
didodecyl-3-methoxy-4-hydroxy benzylidene malonate,
dipalmitoyl-3-methoxy-4-hydroxy benzylidene malonate, or
di-isopropyl-3-methoxy-4-hydroxy benzylidene malonate.

6. A method as in claim 1 wherein the UV-A organic sunscreen is a dibenzoyl methane derivative.

7. A method as in claim 6 wherein the dibenzoyl methane derivative. is selected from the group consisting of;
2-methyldibenzoylmethane, 4-methyldibenzoylmethane, 4-isopropyldibenzoylmethane, 4-tert-butyldibenzoylmethane, 2,4-dimethyldibenzoylmethane, 2,5-dimethyldibenzoylmethane, 4,4′-diisopropyldibenzoylmethane, 4,4′-dimethoxydibenzoylmethane, 4-tert-butyl-4′-methoxydibenzoylmethane, 2-methyl-5-isopropyl-4′-methoxydibenzoylmethane, 2-methyl-5-tert-butyl-4′-methoxydibenzoylmethane, 2,4-dimethyl-4′-methoxydibenzoylmethane, and/or 2,6-dimethyl-4-tert-butyl-4′-methoxydibenzoylmethane).

8. A method according to claim 1, wherein the UV-B sunscreen is a salicylate derivative of the general formula X or a methoxycinnamate derivatives of the general formula Y or a cyanoacrylate derivatives of the general formula Z below. 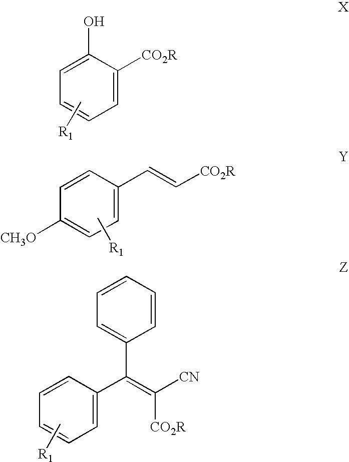
wherein R is linear or branched C₁ to C₂₀ alkyl and R₁ is hydrogen or a linear or branched C₁ to C₈ alkyl.

9. A method according to claim 8 wherein the salicylate derivatives is octyl salicylate, homosalate, triethane amine (TEA)-salicylate or diethylamine (DEA)-salicylate.

10. A method according to claim 8 wherein the methoxy cinnamate derivative is octyl 4-methoxycinnamate, isoamyl-4-methoxycinnamate or ethoxylethyl-4-methoxycinnamte.

11. A method according to claim 8 wherein the cyanoacrylate derivative is octocrylene.

12. A method according to claim 1, wherein the photosensitive compound is a colorant, antioxidant, flavoring agent, vitamin, U.V. filter, fragrance, polymeric thickening agent, oil, wax or photosensitive inorganic compound.

13. A method according to claim 1, wherein the dialkylbenzalmalonate compound is Di-2-ethylhexyl 3,5 dimethoxy 4 hydroxy benzylidene malonate, Isoamyl alpha acetyl-3,5 dimethoxy 4 hydroxy benzylidene malonate, or Bis-N[3′-(N,N dimethylamino)propyl]-3,5-dimethoxy-4 hydroxybenzylidene malonamide bis ethyl sulfate.

14. A method as in claim 1, wherein said photosensitive compound(s) is combined with at least three photostabilizing compounds within a cosmetic composition, personal care product or household product, wherein the total weight of the three photostabilizing agents is used in an amount of about 0.001 to 10.0 wt %, based on the total weight of the cosmetic composition, personal care product or household product.

15. A method as in claim 1, comprising the additional step of transferring the combination of at least one photosensitive compound and the three photostabilizing agents to a cosmetic composition, personal care product or household product within a container for retail sale.

16. A method according to claim 1, wherein the photosensitive compound is a carotenoid, tocopherol, guaiazulene, vanillin, menthylanthranillate, Ubiquinone or Azulene.

17. A method as in claim 16, wherein the Azulene photosensitive poly-unsaturated color compounds is azulene, guaiazulene, or guaiol.

18. A method as in claim 1, wherein the photosensitive compound is Lycopene, Zeaxanthine, Cantaxanthine, α-, β- γ- & δ-Carotenes, Astacin, Astaxanthin, Chrysanthemaxanthin, Torularhodin, Violaxanthin, Capsanthin or Capsorubin.

19. A method as in claim 1, wherein the photosensitive poly-unsaturated color compounds is a photosensitive dye or organic pigment.

20. A method as in claim 19, wherein the photosensitive organic pigment is a curcuminoid.

21. A method as in claim 20, wherein the curcuminoid is curcumin, Cassumunin A, Cassumunin B, demethoxycurcumin or bisdemethoxycurcumin.

22. A method according to claim 1, wherein said photosensitive compound is a fragrance that is combined with three photostabilizing agents used in an amount of about 0.001 to 10.0 wt %, based on the total weight of the resultant composition, and wherein the fragrance is

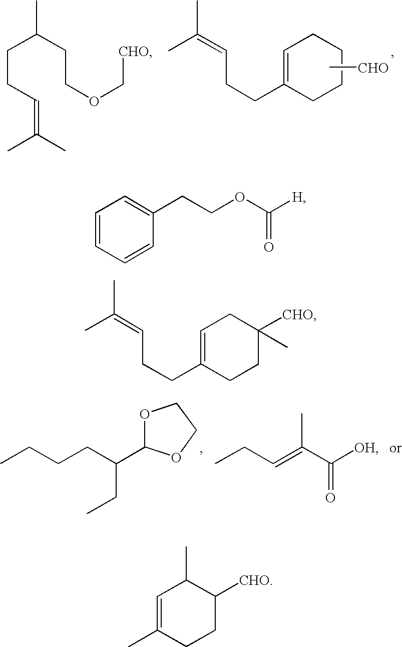

23. A product which is cosmetic composition, personal care product or household product which contains at least one photosensitive compound which is either a color compound, antioxidant, a fragrance compound, a flavor compound or a combination thereof,
where said photosensitive compound is stabilized by the method of claim 1.

24. A product of claim 23, wherein the photosensitive compound is an organic pigment, which is a curcuminoid, selected from curcumas, Cassumunin A, Cassumunin B, demethoxycurcumin or bisdemethoxycurcumin.

25. A product of claim 23, wherein the photosensitive compound is a carotenoid, tocopherol, guaiazulene, vanillin, menthylanthranillate, ubiquinone, azulene, guaiazulene, or guaiol.
